(19) United States
(12) Patent Application Publication
(10) Pub. No.: US 2025/0289014 A1
(43) Pub. Date: Sep. 18, 2025

(54) SPRAY NOZZLE

(71) Applicant: Murata Manufacturing Co., Ltd., Kyoto (JP)

(72) Inventors: Mitsushiro TERASAWA, Kyoto (JP); Kouki EJIRI, Kyoto (JP)

(21) Appl. No.: 19/080,155

(22) Filed: Mar. 14, 2025

(30) Foreign Application Priority Data
Mar. 18, 2024 (JP) 2024-042475

(51) Int. Cl. B05B 7/24

(52) U.S. Cl. CPC B05B 7/2491

(57) ABSTRACT

A spray nozzle includes a nozzle body and a cover member. An outer surface of the nozzle body and an inner surface of the cover member define a suction passage. The nozzle body defines a gas injection passage and a liquid ejection passage. The liquid ejection port is located on a positive direction side of the gas injection port. The cover member includes a cover portion, in contact with an upper surface of the nozzle body facing the positive direction side on the positive direction side of the upper surface, that is exposed to the outside. Of end faces of the cover portion that face the gas injection port, the end face located closest to a negative direction side is located outward of the edge of the gas injection port in a direction orthogonal to the positive direction.

### CROSS REFERENCE TO RELATED APPLICATION

This application claims priority from Japanese Patent Application No. 2024-042475 filed on Mar. 18, 2024. The content of this application is incorporated herein by reference in its entirety.

### BACKGROUND OF THE DISCLOSURE

### 1. Field of the Disclosure

The present disclosure relates to a spray nozzle.

### 2. Description of the Related Art

The spray nozzle described in Japanese Unexamined Patent Application Publication No. 2-46862 includes a gas injection passage and a liquid ejection passage. A gas is injected from the gas injection passage. A liquid can circulate through the liquid ejection passage. The central axis of the liquid ejection passage is orthogonal to the central axis of the gas injection passage. The gas injection passage and the liquid ejection passage are integrally formed with each other. In this spray nozzle, the liquid is sucked through the liquid ejection passage as the gas is injected. Then, the sucked liquid is ejected to the outside of the liquid ejection passage. The ejected liquid is atomized by colliding with the gas.

### BRIEF SUMMARY OF THE DISCLOSURE

When the liquid ejection passage and the gas injection passage are integrally formed with each other as in the spray nozzle described in Japanese Unexamined Patent Application Publication No. 2-46862, there may be cases in which the inside of the liquid ejection passage cannot be properly cleaned when an attempt is made to clean the inside. For example, when the spray nozzle is formed of a plurality of components such that these components can be separated from each other, ease of cleaning can be improved. However, when the spray nozzle can be separated into a plurality of components, there is a risk that the individual components become misaligned during use.

To solve the problem described above, according to the present disclosure, there is provided a spray nozzle including: a nozzle body connected to a pump; and a cover member, attachable to and detachable from the nozzle body the cover member, that covers an outer surface of the nozzle body, in which the outer surface of the nozzle body and an inner surface of the cover member define a suction passage through which a liquid circulates, the nozzle body defines a gas injection passage through which a liquid pressurized and fed from the pump circulates and a liquid ejection passage connected to the suction passage on a downstream side, in a cross section including a gas injection port that is an opening of the gas injection passage on a downstream side and a liquid ejection port that is an opening of the liquid ejection passage on a downstream side, when a direction from the gas injection port to the liquid ejection port is a positive direction and a direction opposite to the positive direction is a negative direction, the cover member includes a cover portion, in contact with, on a positive direction side, an upper surface of the nozzle body facing the positive direction side, that is exposed to an outside, and, of end faces of the cover portion that face the gas injection port, an end face located closest to a negative direction side is located outward of an edge of the gas injection port in a direction orthogonal to the positive direction.

The structure described above makes cleaning of the suction passage easier. In addition, the structure described above can suppress the cover member from being misaligned so as to float up from the nozzle body.

### BRIEF DESCRIPTION OF THE SEVERAL VIEWS OF THE DRAWINGS

FIG. 1 is a perspective view illustrating a nebulizer;

FIG. 2 is a schematic end view of a tank unit, a spray nozzle, and a discharge unit;

FIG. 3 is a schematic end view of the spray nozzle;

FIG. 4 is a schematic end view of a spray nozzle according to a modification;

FIG. 5 is a schematic end view of a spray nozzle according to a modification;

FIG. 6 is a schematic end view of a spray nozzle according to a modification; and

FIG. 7 is a schematic end view of a spray nozzle according to a modification.

### DETAILED DESCRIPTION OF THE DISCLOSURE

A spray nozzle according to one embodiment will be described below. In the following embodiment, the spray nozzle is applied to a nebulizer. It should be noted that components in the drawings may be enlarged for ease of understanding. The dimensional ratios of components may differ from those in an actual object or differ between different drawings.

### Overall Structure of Nebulizer

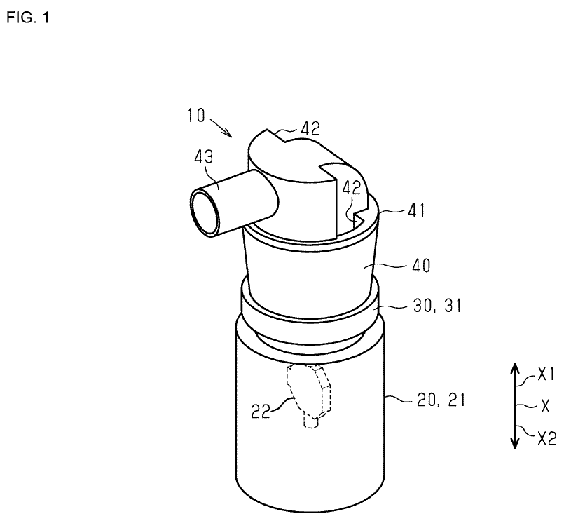

As illustrated in FIG. 1, a nebulizer 10 includes a pump unit 20, a tank unit 30, and a discharge unit 40.

The pump unit 20 includes a pump case 21 and a pump 22. The pump case 21 is a substantially cylindrical case. The pump 22 is located in the pump case 21. The pump 22 is a so-called piezoelectric pump. The pump 22 includes a piezoelectric element and a diaphragm, which are not illustrated. The piezoelectric element is made of piezoelectric ceramic. In addition, the pump 22 can pressurize and feed air by repeatedly deflecting a diaphragm through vibration of the piezoelectric element. That is, the pump 22 can pressurize and feed a gas.

Here, it is assumed that an axis parallel to a central axis of the pump case 21 is a specific axis X. In addition, it is assumed that one direction along the specific axis X is a positive direction X1, the direction opposite to the positive direction X1 is a negative direction X2.

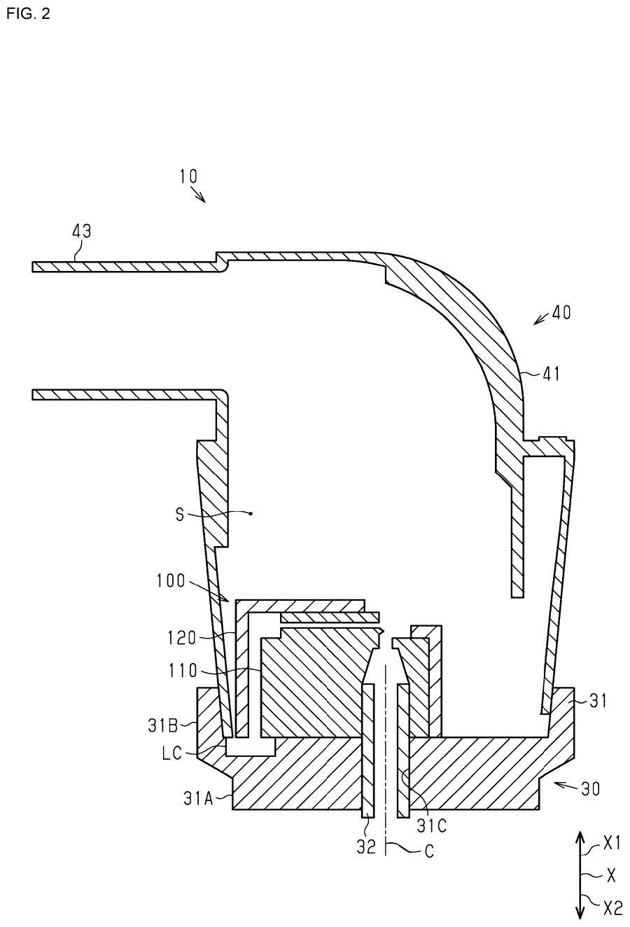

The tank unit 30 is attached to an end portion of the pump unit 20 in the positive direction X1. As illustrated in FIG. 2, the tank unit 30 includes a tank body 31 and a pipe 32.

The tank body 31 is substantially bottomed-cylindrical. An end portion of the tank body 31 in the negative direction X2 is blocked. That is, the tank body 31 includes a bottom wall 31A and a side wall 31B. The tank body 31 extends in a direction along the specific axis X as a whole.

The bottom wall 31A is disk-shaped as a whole. The outer diameter of a portion including an end of the bottom wall 31A in the negative direction X2 is smaller than the outer diameter of an end in the positive direction X1 of the bottom wall 31A. The side wall 31B rises in the positive direction X1 from the outer edge of the surface of the bottom wall 31A that faces the positive direction X1 side. The side wall 31B extends over the entire area of the outer edge of the bottom wall 31A.

The tank body 31 has a through-hole 31C. The through-hole 31C passes through the bottom wall 31A in the direction along the specific axis X. The through-hole 31C is located substantially at the center of the bottom wall 31A.

In addition, the tank body 31 has a liquid storage chamber LC. The liquid storage chamber LC is recessed toward the negative direction X2 side from the bottom wall 31A of the tank body 31. The position of the liquid storage chamber LC differs from that of the through-hole 31C. Specifically, the liquid storage chamber LC is closer to the outer edge of the bottom wall 31A as viewed from the through-hole 31C and is adjacent to the side wall 31B in a direction orthogonal to the specific axis X.

The pipe 32 is cylindrical. The outer diameter of the pipe 32 is slightly smaller than the inner diameter of the through-hole 31C of the tank body 31. The pipe 32 is fitted to the through-hole 31C. In addition, the dimension of pipe 32 in the direction along the specific axis X is larger than the dimension of the bottom wall 31A in the direction along the specific axis X. A portion of the pipe 32 on the positive direction X1 side projects to the positive direction X1 side from the bottom wall 31A. In addition, a portion of the pipe 32 on the negative direction X2 side projects to the negative direction X2 side from the bottom wall 31A. An end of the pipe 32 in the negative direction X2 is connected to the pump 22. That is, the gas pressurized and fed from the pump 22 is pressurized and fed to the positive direction X1 side of the tank unit 30 through the pipe 32.

As illustrated in FIG. 1, the discharge unit 40 is attached to a portion of the tank unit 30 on the positive direction X1 side. The discharge unit 40 includes a case body 41, intake holes 42, and a discharge pipe 43.

As illustrated in FIG. 2, the case body 41 is a cylinder having openings at both ends. The case body 41 extends in the direction along the specific axis X as a whole. The opening of the case body 41 on the negative direction X2 side is fitted to the opening of the tank body 31. Accordingly, an internal space S is defined by the tank body 31 and the case body 41. The opening of the case body 41 on the positive direction X1 side faces a direction that intersects the specific axis X.

As illustrated in FIG. 1, the intake holes 42 are through-holes that pass through a surface of the case body 41 that faces the positive direction X1 side. That is, the intake holes 42 connect the inner space S and the outside of the case body 41 to each other. Each of the intake holes 42 is provided on either side of the central axis of the case body 41.

As illustrated in FIG. 2, the discharge pipe 43 is connected to the opening of the case body 41 on the positive direction X1 side. Specifically, the discharge pipe 43 extends in the direction orthogonal to the specific axis X. The inside of the discharge pipe 43 is connected to the inner space S of the case body 41.

### Spray Nozzle

As illustrated in FIG. 2, the nebulizer 10 includes a spray nozzle 100. The spray nozzle 100 is connected to an end portion of the pipe 32 in the positive direction X1. The spray nozzle 100 is located on the positive direction X1 side of the bottom wall 31A of the tank body 31. That is, the spray nozzle 100 is located in the inner space S. The spray nozzle 100 is a member for atomizing a liquid, such as a chemical solution. The liquid atomized by the spray nozzle 100 is discharged from the discharge pipe 43 through the inner space S.

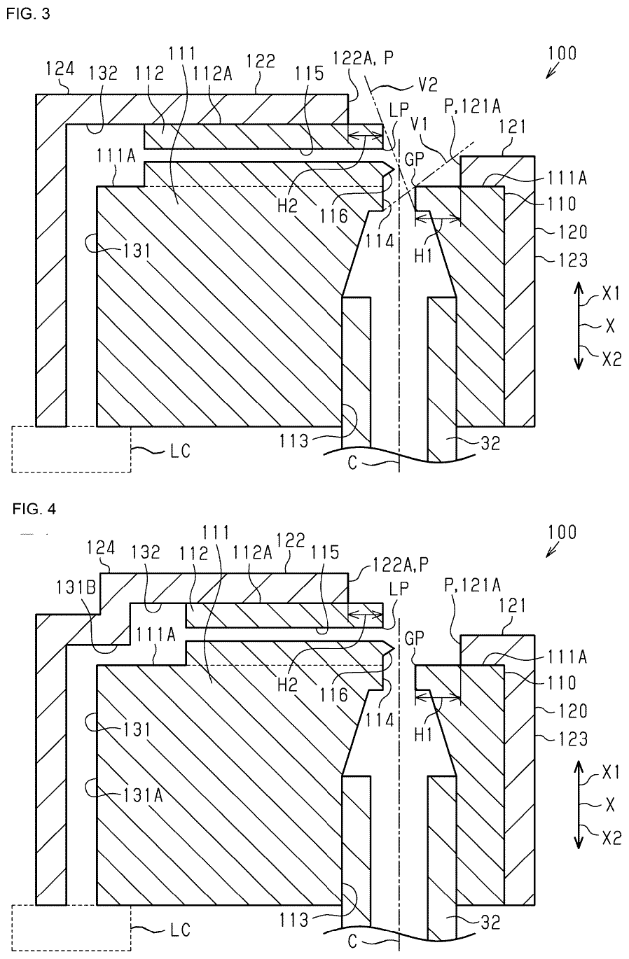

As illustrated in FIG. 3, the spray nozzle 100 includes a nozzle body 110 and a cover member 120. The material of the nozzle body 110 is polyethylene, ABS resin, polycarbonate, or the like. In addition, the material of the cover member 120 is also the same.

The nozzle body 110 includes mainly a first portion 111 and a second portion 112. The first portion 111 has a cylindrical shape that extends in the direction along the specific axis X as a whole. The second portion 112 has a columnar shape that extends in the direction along the specific axis X. The second portion 112 projects from a surface of the first portion 111 that faces the positive direction X1 side. In addition, the dimension of the second portion 112 in the direction orthogonal to the specific axis X is smaller than the dimension of the first portion 111 in the direction orthogonal to the specific axis X. Accordingly, there is a step at the boundary between the second portion 112 and the first portion 111. In addition, the second portion 112 and the first portion 111 are integrally formed with each other. Accordingly, a clear boundary is not often present between the first portion 111 and the second portion 112 in the nozzle body 110. It should be noted that the boundary between the first portion 111 and the second portion 112 is illustrated virtually by the dashed line in FIG. 3.

The nozzle body 110 includes a connection passage 113, a gas injection passage 114, and a liquid ejection passage 115. In other words, in the nozzle body 110, the connection passage 113, the gas injection passage 114, and the liquid ejection passage 115 are defined.

The connection passage 113 extends in the direction along the specific axis X as a whole in the first portion 111 of the nozzle body 110. An end of the connection passage 113 in the negative direction X2 is open in the surface of the first portion 111 of the nozzle body 110 that faces the negative direction X2 side. The inner diameter of the connection passage 113 at the end in the negative direction X2 is slightly larger than the outer diameter of the pipe 32 described above. In addition, the end of the connection passage 113 in the negative direction X2 is fitted onto the pipe 32. That is, the connection passage 113 is connected to the pipe 32. In other words, the nozzle body 110 is connected to the pump 22 through the pipe 32. The flow path sectional area of a portion including an end of the connection passage 113 in the positive direction X1 decreases toward the positive direction X1 side.

The gas injection passage 114 extends in the direction along the specific axis X in the first portion 111 of the nozzle body 110. The gas injection passage 114 is a substantially cylindrical passage. An end of the gas injection passage 114 in the negative direction X2 is connected to the end of the connection passage 113 in the positive direction X1. An end of the gas injection passage 114 in the positive direction X1 is open in a surface of the first portion 111 of the nozzle body 110 that faces the positive direction X1 side. That is, the gas injection passage 114 is open at a location on the surface of the first portion 111 that faces the positive direction X1 side at which the second portion 112 is not present. Accordingly, the gas injection passage 114 and the connection passage 113 pass through the first portion 111 of the nozzle body 110 in the direction along the specific axis X as a whole. In addition, the gas pressurized and fed from the pump 22 circulates through the gas injection passage 114 through the pipe 32 and connection passage 113. Then, the gas injected from the gas injection passage 114 reaches the inner space S. It should be noted that the maximum flow path sectional area of the gas injection passage 114 is smaller than the maximum flow path sectional area of the connection passage 113. The flow path sectional area is the area of a passage in a cross section orthogonal to the flow direction of a gas or a liquid. In addition, the flow direction of a gas or a liquid is a flow direction determined when the pump 22 is driven.

Here, it is assumed that the opening of the gas injection passage 114 on the downstream side is referred to as a gas injection port GP. That is, the gas injection port GP is an opening of the gas injection passage 114 located on the positive direction X1 side. In the present embodiment, the gas injection port GP is circular in plan view. In addition, of the directions along the central axis C of the gas injection port GP, the direction faced by the gas injection port GP is a positive direction, and the direction opposite to the positive direction is a negative direction.

The central axis C of the gas injection port GP is determined as described below. First, the orientation of the line of sight that maximizes the apparent area of the range surrounded by the outer edge of the gas injection port GP from the inside of the gas injection passage 114 is determined. Next, the geometric center of the shape of the outer edge of the gas injection port GP is determined in accordance with the determined orientation of the line of sight. Then, the axis, parallel to the orientation of the line of sight, that passes through the geometric center is determined to be a central axis C of the gas injection port GP.

It should be noted that the central axis C is parallel to the specific axis X in the present embodiment. Accordingly, the direction faced by the gas injection port GP coincides with the positive direction X1. In addition, the direction opposite to the direction faced by the gas injection port GP coincides with the negative direction X2.

The liquid ejection passage 115 extends in the direction orthogonal to the specific axis X as a whole in the second portion 112 of the nozzle body 110. That is, the liquid ejection passage 115 is located on the positive direction X1 side of the gas injection port GP. The liquid ejection passage 115 is a substantially cylindrical passage. The first end of the liquid ejection passage 115 is open in a surface facing the gas injection port GP of the outer surfaces of the second portion 112. The second end of the liquid ejection passage 115 is open in a surface facing away from the gas injection port GP of the outer surfaces of the second portion 112. That is, the liquid ejection passage 115 passes through the second portion 112 in a direction orthogonal to the central axis C.

Here, the opening of the liquid ejection passage 115 on the downstream side is referred to as a liquid ejection port LP. The liquid ejection port LP is an opening facing the gas injection port GP of the openings of the liquid ejection passage 115. The liquid ejection port LP is circular in plan view in the present embodiment. The liquid ejection port LP is located on the positive direction X1 side of the gas injection port GP.

In addition, a cross-sectional view of the spray nozzle 100 according to the present embodiment taken along a cross section including the gas injection port GP and the liquid ejection port LP is assumed. At this time, an example of the direction from the gas injection port GP to the liquid ejection port LP is the positive direction X1. Accordingly, the direction orthogonal to the central axis C coincides with the direction orthogonal to the positive direction X1. In addition, the direction along the central axis C coincides with the direction along the positive direction X1 or the negative direction X2. Similarly, intersecting the central axis C means intersecting the positive direction X1.

The direction faced by the liquid ejection port LP intersects the central axis C of the gas injection port GP. It should be noted that the direction faced by the liquid ejection port LP can be determined by the orientation of the central axis of the liquid ejection port LP. The central axis of the liquid ejection port LP can be determined in the same way as the central axis C of the gas injection port GP described above. In addition, of the directions along the central axis of the liquid ejection port LP, the direction from the liquid ejection port LP to the gas injection port GP is assumed to be the direction faced by the liquid ejection port LP.

In addition, the nozzle body 110 includes a projection 116. The projection 116 has a conical shape tapered toward the tip. The projection 116 projects toward the gas injection port GP from the side surface facing the gas injection port GP of the outer surfaces of the second portion 112. Specifically, the projection 116 is located on the negative direction X2 side of the liquid ejection port LP. It should be noted that the projection 116 suppresses the liquid from dropping from the liquid ejection port LP when the driving of the pump 22 is stopped.

Here, it is assumed that the surfaces facing the positive direction X1 side of the outer surfaces of the nozzle body 110 are upper surfaces. However, the upper surfaces are not limited to surfaces orthogonal to the positive direction X1. The upper surfaces of the nozzle body 110 are visible surfaces when the nozzle body 110 is viewed in the negative direction X2.

The nozzle body 110 has a first upper surface 111A and a second upper surface 112A as the upper surfaces. The first upper surface 111A is a surface, located at the same position as the end in the positive direction X1 of the gas injection port GP in the direction along the central axis C of the gas injection port GP, that is orthogonal to the central axis C of the gas injection port GP. The first upper surface 111A is planar. In the present embodiment, the first upper surface 111A is a surface that does not have the second portion 112 of the surfaces of the first portion 111 that face the positive direction X1 side.

In addition, the second upper surface 112A is located on the positive direction X1 side of the liquid ejection passage 115. In the present embodiment, the second upper surface 112A is a surface of the second portion 112 that faces the positive direction X1 side.

The cover member 120 is attachable to and detachable from the nozzle body 110. The cover member 120 attached to the nozzle body 110 will be described below.

As illustrated in FIG. 3, the cover member 120 covers the outer surface of the nozzle body 110. Specifically, the cover member 120 covers the entire side surface of the nozzle body 110 and at least a portion of the upper surface of the nozzle body 110. It should be noted that “the cover member 120 covers the outer surface” includes the case in which the cover member 120 is in direct contact with the nozzle body 110 and the case in which the cover member 120 faces the nozzle body 110 with a space therebetween.

The cover member 120 has a substantially cylindrical shape with a closed end on the positive direction X1 side. Specifically, the cover member 120 includes a cylindrical side-wall portion 123 and a top-wall portion 124 that blocks an opening of the side-wall portion 123 on the positive direction X1 side. A portion of the top-wall portion 124 in contact with the upper surface of the nozzle body 110 on the positive direction X1 side of the upper surface is the cover portion. The cover portion is exposed to the outside of the spray nozzle 100, that is, to the inner space S on the positive direction X1 side.

The cover portion partially covers the first upper surface 111A and the second upper surface 112A of the nozzle body 110. As described above, a step is present between the first portion 111 and the second portion 112 of the nozzle body 110. The cover portion of the cover member 120 also has a step shape to reflect the step described above. It should be noted that, in the following description, a portion of the cover portion in contact with the first upper surface 111A is referred to as a first cover portion 121, and a portion in contact with the second upper surface 112A is referred to as a second cover portion 122. That is, the cover portion includes the first cover portion 121 and the second cover portion 122.

The cover portion has a cavity P. The central axis of the cavity P is aligned with the central axis C of the gas injection port GP. The gas injection port GP can be seen through the cavity P of the cover member 120 as viewed in the negative direction X2.

The spray nozzle 100 includes a suction passage 131 and a liquid pool 132. The suction passage 131 and the liquid pool 132 are passages defined by the outer surface of the nozzle body 110 and the inner surface of the cover member 120, respectively. The suction passage 131, the liquid pool 132, and the liquid ejection passage 115 are passages through which the liquid circulates.

The suction passage 131 is defined by the inner surface of the cover member 120 and the side surface of the first portion 111 of the nozzle body 110. That is, the suction passage 131 extends in the direction along the specific axis X. The maximum flow path sectional area of the suction passage 131 is larger than the maximum flow path sectional area of the liquid ejection passage 115. The end of the suction passage 131 in the negative direction X2 is connected to the liquid storage chamber LC of the tank unit 30. It should be noted that, in FIG. 3, the liquid storage chamber LC is illustrated virtually by the dashed line.

The liquid pool 132 is defined by the inner surface of the cover member 120, the first upper surface 111A of the nozzle body 110, and the side surface of the second portion 112 near the opening on the upstream side of the liquid ejection passage 115. The liquid pool 132 is a substantially rectangular space. The liquid pool 132 is connected to the end of the suction passage 131 in the positive direction X1. In addition, the liquid pool 132 is connected to the opening of the liquid ejection passage 115 on the upstream side. Accordingly, the liquid pool 132 is located between the suction passage 131 and the liquid ejection passage 115. The maximum flow path sectional area of the liquid pool 132 is larger than the maximum flow path sectional area of the suction passage 131. In addition, the maximum flow path sectional area of the liquid pool section 132 is larger than the maximum flow path sectional area of the liquid ejection passage 115.

In the flow channel structure described above, the liquid stored in the liquid storage chamber LC flows through the suction passage 131, the liquid pool 132, and the liquid ejection passage 115 in this order. Then, the liquid is ejected to the outside through the liquid ejection port LP of the liquid ejection passage 115. That is, the liquid ejection passage 115 is connected to the suction passage 131 on the downstream side in the flow direction of the liquid.

Positional Relationship between Cover Portion and Gas Injection port

As illustrated in FIG. 3, the end face of the first cover portion 121 that faces the gas injection port GP is referred to as a first end face 121A. In addition, the end face of the second cover portion 122 that faces the gas injection port GP is referred to as a second end face 122A. In the present embodiment, the first end face 121A is a portion of the inner peripheral surface of the cavity P of the cover portion. Similarly, the second end face 122A is a portion of the inner peripheral surface of the cavity P of the cover portion. It should be noted that the first upper surface 111A is located on the negative direction X2 side of the second upper surface 112A as described above. Accordingly, of the end faces of the cover portions that face the gas injection port GP, the end face located closest to the negative direction X2 side is the first end face 121A.

The first end face 121A is located outward of the edge of the gas injection port GP in the direction orthogonal to the central axis C of the gas injection port GP. In addition, the second end face 122A is located outward of the edge of the gas injection port GP in the direction orthogonal to the central axis C of the gas injection port GP. That is, the entire areas of the end faces of the cover portion that face the gas injection port GP are located outward of the edge of the gas injection port GP in the direction orthogonal to the central axis C of the gas injection port GP.

Here, it is assumed that a first virtual straight line V1 that passes through an end of the gas injection passage 114 in the negative direction X2 and an edge of the first upper surface 111A closest to the gas injection port GP and crosses the central axis C of the gas injection port GP is drawn. The entire area of the first end face 121A is located outward of the first virtual straight line V1 in the direction orthogonal to the central axis C of the gas injection port GP.

Similarly, it is assumed that a second virtual straight line V2 that passes through an end of the gas injection passage 114 in the negative direction X2 and an edge of the second upper surface 112A closest to the gas injection port GP and crosses the central axis C of the gas injection port GP is drawn. The entire area of the second end face 122A is located outward of the second virtual straight line V2 in the direction orthogonal to the central axis C of the gas injection port GP. It should be noted that the first virtual straight line V1 and the second virtual straight line V2 have been used as examples for description, but the positional relationship described above is not limited to only these two virtual straight lines. In the present embodiment, the entire areas of the end faces of the cover portions that face the gas injection port GP are located outward of any virtual straight line that passes through an end of the gas injection passage 114 in the negative direction X2 and an edge of the upper surface of the nozzle body 110 closest to the gas injection port GP and crosses the central axis C of the gas injection port GP.

It is assumed that the shortest dimension from the gas injection port GP to the first end face 121A in the direction orthogonal to the central axis C is a first dimension H1. The first dimension H1 is larger than the maximum dimension of the gas injection port GP. It should be noted that the maximum dimension of the gas injection port GP is the diameter of the gas injection port GP. In addition, it is assumed that the shortest dimension from the gas injection port GP to the second end face 122A in the direction orthogonal to the central axis C is a second dimension H2. The second dimension H2 is larger than the maximum dimension of the gas injection port GP.

### Operation of Present Embodiment

When the pump 22 is driven, the gas is pressurized and fed from the pump 22. The gas to be pressurized and fed circulates through the connection passage 113 and the gas injection passage 114 through the pipe 32. Then, the gas is injected into the inner space S through the gas injection port GP.

The gas injected through the gas injection port GP passes near the liquid ejection port LP. As a result, a negative pressure is caused near the liquid ejection port LP. On the other hand, an end of the suction passage 131 in the negative direction X2 is connected to the liquid storage chamber LC. Accordingly, the negative pressure described above causes the liquid, such as a chemical solution, stored in the liquid storage chamber LC to enter the suction passage 131, pass through the liquid pool 132 and the liquid ejection passage 115, and be ejected through the liquid ejection port LP.

The liquid ejected through the liquid ejection port LP collides with the gas injected through the gas injection port GP and become fine liquid droplets. A flow of the gas pressurized and fed from the pump 22 causes the fine liquid droplets to pass through the inner space S and the inside of the discharge pipe 43 and be discharged to the outside of the nebulizer 10.

### Effects of Present Embodiment

(1) According to the embodiment described above, the cover member 120 is attachable to and detachable from the nozzle body 110. Accordingly, by the nozzle body 110 being separated from the cover member 120, the suction passage 131 defined by the cover member 120 and the nozzle body 110 can be easily cleaned. Accordingly, even if clogging of the suction passage 131 occurs due to a solidified chemical solution or the like, the clogging can be easily eliminated in the structure described above.

In addition, in the present embodiment, of the end faces of the cover portions that face the gas injection port GP, the first end face 121A located closest to the negative direction X2 side is located outward of the edge of the gas injection port GP in the direction orthogonal to the central axis C of the gas injection port GP. In this structure, the gas injected through the gas injection port GP does not collide with the first cover portion 121 from the negative direction X2 side. That is, the gas injected through the nozzle body 110 can be prevented from colliding with a portion of the cover member 120 closest to the gas injection port GP from the negative direction X2 side to a positive direction X1 side. Accordingly, in this structure, the cover member 120 can be suppressed from being misaligned so as to float up from the nozzle body 110. In addition, if the cover member 120 can be suppressed from being misaligned as described above, the suction passage 131 defined by the inner surface of the cover member 120 and the outer surface of the nozzle body 110 can be prevented from unintentionally communicating with the inner space S or the shape of the flow channel of the suction passage 131 can be prevented from changing from a designed shape.

(2) In the embodiment described above, the spray nozzle 100 includes the liquid pool 132 that has a maximum flow path sectional area larger than the maximum flow path sectional area of the suction passage 131. In this structure, the spray nozzle 100 can store, in the liquid pool 132, the liquid that has passed through the suction passage 131. Even if, for example, the supply of the liquid from the liquid storage chamber LC is temporarily stopped for some reason, liquid droplets discharged from the nebulizer 10 are less likely to be interrupted because the liquid stored in the liquid pool 132 is ejected.

(3) In the embodiment described above, the liquid pool 132 is defined by the outer surface of the nozzle body 110 and the inner surface of the cover member 120. In this structure, by the nozzle body 110 being separated from the cover member 120, the liquid pool 132 can be easily cleaned.

(4) In the embodiment described above, the cover member 120 includes the first cover portion 121 in contact with the first upper surface 111A. Since the first upper surface 111A is in contact with the first cover portion 121, the close contact force between the nozzle body 110 and the cover member 120 is improved. Accordingly, misalignment between the nozzle body 110 and the cover member 120 during the use of the nebulizer 10 is less likely to occur.

(5) In the embodiment described above, the cover member 120 includes the second cover portion 122 in contact with the second upper surface 112A. Since the second upper surface 112A is in contact with the second cover portion 122, the close contact force between the nozzle body 110 and the cover member 120 is further improved. Accordingly, the effect of preventing the misalignment between the nozzle body 110 and the cover member 120 during the use of the nebulizer 10 is obtained.

(6) In the present embodiment, the entire areas of the end faces of the cover portions that face the gas injection port GP are located outward of the edge of the gas injection port GP in the direction orthogonal to the central axis C of the gas injection port GP. In other words, the second cover portion 122 located on the positive direction X1 side of the first cover portion 121 is also located outward of the edge of the gas injection port GP. In this structure, the cover member 120 can be more notably suppressed from being misaligned so as to float up from the nozzle body 110.

(7) If the gas injected through the gas injection port GP circulates linearly, the region inside the virtual straight line in the embodiment described above is a region through which the gas can circulate. In the embodiment described above, the entire areas of the end faces of the cover portions that face the gas injection port GP are located outward of the virtual straight lines described above. Since the entire areas of the end faces of the cover portions that face the gas injection port GP are located outside the region through which the gas can circulate in this structure, the cover portions are less likely to collide with the gas.

(8) If the liquid ejection port LP is formed in the cover member 120, precise alignment between the liquid ejection port LP and the gas injection port GP is necessary when the cover member 120 is attached to the nozzle body 110. In the embodiment described above, the nozzle body 110 has the gas injection port GP and the liquid ejection port LP. That is, the gas injection port GP and the liquid ejection port LP are formed of one member. Accordingly, in the structure described above, precise alignment as described above is not necessary.

(9) In the embodiment described above, the first dimension H1 is larger than the maximum dimension of the gas injection port GP. In addition, the second dimension H2 is larger than the maximum dimension of the gas injection port GP. As described above, since the end faces of the cover portion are sufficiently away from the gas injection port GP in the direction orthogonal to the central axis C, the gas injected through the gas injection port GP and the gas emitted from the cover portion are less likely to collide with each other.

### Modification

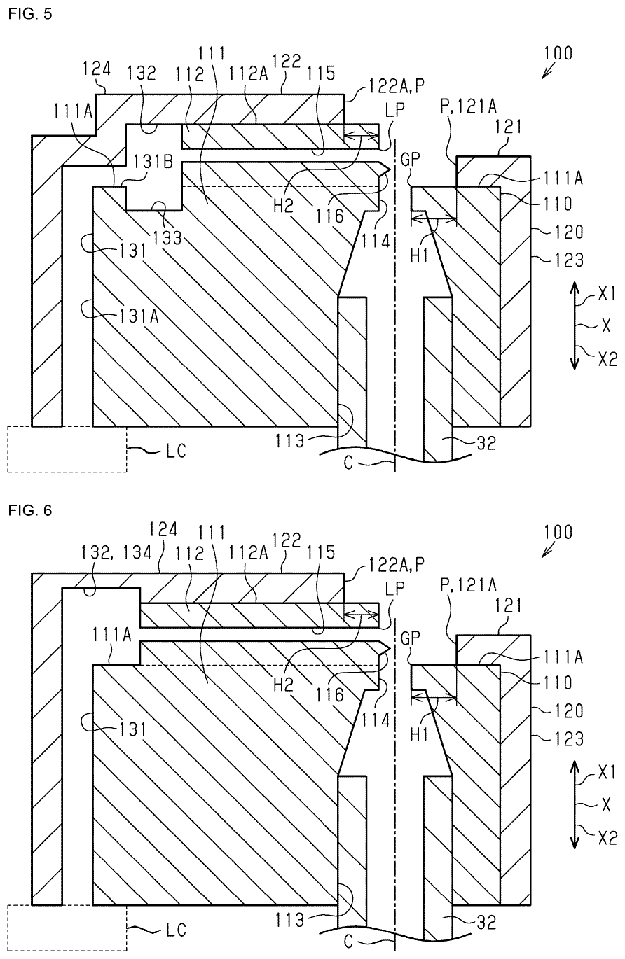

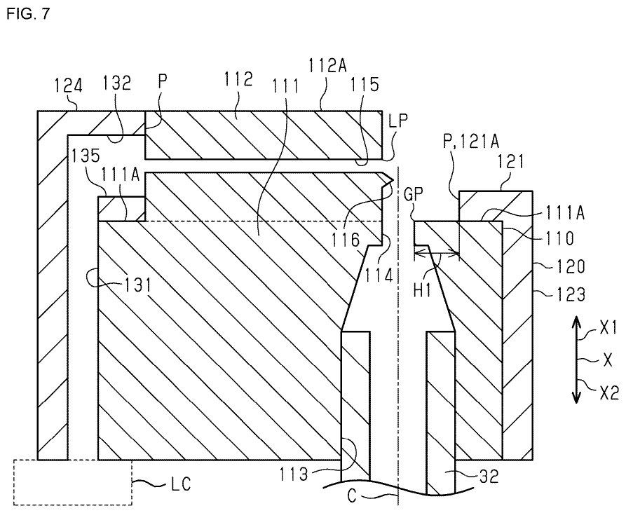

The embodiment described above and the following modification can be implemented by being combined with each other within a technically non-contradictory range.
In the embodiment described above, the application of the spray nozzle 100 is not limited to the nebulizer 10. The technology concerning the spray nozzle 100 described above is applicable to a device for atomizing a liquid.
In the embodiment described above, the pump 22 is not limited to a piezoelectric pump. For example, the pump 22 may be a rotary pump or the like. Depending on the type of the pump 22, the pump case 21 and the pipe 32 may be connected to each other through a hose or the like.
In the embodiment described above, the shapes and structures of the pump unit 20, the tank unit 30, and the discharge unit 40 may be changed as appropriate.
In the embodiment described above, the liquid storage chamber LC need not be defined by the tank body 31. For example, the liquid storage chamber LC may be defined by the spray nozzle 100 or may be a liquid chamber that differs from the member described in the embodiment described above.
In the embodiment described above, the outer shape of the nozzle body 110 is not limited to the example of the embodiment described above. For example, the nozzle body 110 may have a tapered shape having an outer diameter that decreases in the positive direction X1. In addition, for example, the outer surface of the nozzle body 110 may have a plurality of steps. The same applies to the cover member 120.
In the structure described above, the material of the nozzle body 110 is not limited to the example of the embodiment described above. However, the material of the nozzle body 110 may be a substance having chemical resistance, such as polyethylene, ABS resin, polycarbonate, or the like. The same applies to the material of the cover member 120.
In the embodiment described above, the nozzle body 110 need not include the projection 116. The shape of the liquid ejection port LP can suppress the liquid from dripping through the liquid ejection port LP.
In the embodiment described above, the shape of the liquid ejection port LP and the gas injection port GP is not limited to a circular shape in plan view.
In the embodiment described above, the entire connection passage 113 may be fitted to the pipe 32.
In the embodiment described above, the shape of the gas injection passage 114 is not limited to the example in the embodiment described above. For example, the entire gas injection passage 114 may have a taper shape having a radius that decreases toward the downstream side. The same applies to the liquid ejection passage 115.
In the embodiment described above, the suction passage 131 is not limited to a passage that extends linearly along the specific axis X. In the example illustrated in FIG. 4, the suction passage 131 includes mainly a first suction passage 131A that extends along the specific axis X, and a second suction passage 131B, connected to the first suction passage 131A, that extends in the direction orthogonal to the central axis C. The first suction passage 131A is defined by the inner surface of the cover member 120 and the side surface of the first portion 111 of the nozzle body 110, as in the suction passage 131 according to the embodiment described above. The second suction passage 131B is connected to the downstream side of the first suction passage 131A. The second suction passage 131B is defined by the first upper surface 111A of the first portion 111 of the nozzle body 110 and the inner surface of the cover member 120. That is, in the example illustrated in FIG. 4, the suction passage 131 is also located on the positive direction X1 side of the upper surface of the nozzle body 110. As a result, the liquid pool 132 is located closer to the liquid ejection port LP.
In the embodiment described above, the nozzle body 110 may have a recess on the upper surface. In the example illustrated in FIG. 5, as the example illustrated in FIG. 4, the suction passage 131 includes mainly the first suction passage 131A that extends along the specific axis X, and the second suction passage 131B, connected to the first suction passage 131A, that extends orthogonally to the central axis C. In addition, a portion of the first upper surface 111A of the nozzle body 110 constitutes the bottom surface of the liquid pool 132. In addition, the nozzle body 110 has a recessed portion 133 recessed in the negative direction X2 at a location on the first upper surface 111A at which the bottom surface of the liquid pool 132 is formed. The recessed portion 133 as described above can increase the maximum flow path sectional area and the volume of the liquid pool 132 without changing the positional relationship between the nozzle body 110 and the cover member 120 when the cover member 120 is attached to the nozzle body 110.
In the embodiment described above, the top-wall portion 124 of the cover member 120 may have a recess. In the example illustrated in FIG. 6, the surface of the top-wall portion 124 that faces the negative direction X2 side defines the top surface of the liquid pool 132. In addition, the top-wall portion 124 has a recessed portion 134 recessed in the positive direction X1 at a location at which the top surface of the liquid pool 132 is formed. That is, the thickness dimension of the top-wall portion 124 is partially small. In the recessed portion 134 described above, it is possible to increase the maximum flow path sectional area and the volume of the liquid pool 132 without changing the positional relationship between the nozzle body 110 and the cover member 120 when the cover member 120 is attached to the nozzle body 110.
In the embodiment described above, the cover member 120 need not have the second cover portion 122. For example, in the example illustrated in FIG. 7, the cover member 120 includes a fixed portion 135, adjacent to the opening of the liquid ejection passage 115 of the nozzle body 110 on the upstream side, that is in contact with the first upper surface 111A of the nozzle body 110. On the other hand, the cover member 120 does not cover the second upper surface 112A of the nozzle body 110. In addition, the end face of the cover members 120 that defines the liquid pool 132 and faces the gas injection port GP is in contact with a side surface near the opening of the liquid ejection passage 115 on an upstream side in the second portion 112 of the nozzle body 110. That is, the liquid pool 132 is defined by the fixed portion 135 of the cover member 120, the inner surface facing the fixed portion 135, and the side surface around the opening of the liquid ejection passage 115 on the upstream side in the nozzle body 110. It should be noted that it is possible to prevent the liquid from leaking from the liquid pool 132 by attaching the cover member 120 to the nozzle body 110 while press-fitting the cover member 120 in the direction orthogonal to the central axis C.
In the embodiment described above, the liquid pool 132 need not be defined by the outer surface of the nozzle body 110 and the inner surface of the cover member 120. That is, the liquid pool 132 may be defined by only the cover member 120 or only the nozzle body 110.
In the embodiment described above, the spray nozzle 100 need not include the liquid pool 132. That is, the suction passage 131 may be directly connected to the liquid ejection passage 115.

In the embodiment described above, the maximum flow path sectional area of the liquid ejection passage 115 may be the same as the maximum flow path sectional area of the suction passage 131. However, the maximum flow path sectional area of the suction passage 131 may be four times or more than the maximum flow path sectional area of the liquid ejection passage 115. That is, the maximum flow path sectional area of the liquid pool section 132 may be four times or more than the maximum flow path sectional area of the liquid ejection passage 115 and larger than the maximum flow path sectional area of the suction passage 131.
In the embodiment described above, the cover member 120 only needs to have the cover portion, in contact with the upper surface of the nozzle body 110 facing a positive direction X1 side on the positive direction X1 side of the upper surface, that is exposed to the outside. That is, the cover member 120 need not have the first cover portion 121 or the second cover portion 122 or may have a portion in contact with the upper surface of the nozzle body 110 in addition to the first cover portion 121 and the second cover portion 122 in the embodiment.
In the embodiment described above, the entire areas of the end faces of the cover portions that face the gas injection port GP may be located inward of the virtual straight line or may be in contact with the virtual straight line. For example, in the embodiment described above, while the entire area of the first end face 121A is located outward of the first virtual straight line V1 in the direction orthogonal to the central axis C, the entire area of the second end face 122A may be located inward of the second virtual straight line V2 in the direction orthogonal to the central axis C. The same applies to the opposite case. In addition, if the end face of the cover portion located closest to a negative direction X2 side of the end faces of the cover portions that face the gas injection port GP are located outward of the edge of the gas injection port GP in the direction orthogonal to the central axis C of the gas injection port GP, the entire areas of the end faces of the cover portions that face the gas injection port GP may be located inward of the virtual straight line or may be in contact with the virtual straight line.
In the embodiment described above, the second end face 122A need not be located outward of the edge of the gas injection port GP in the direction orthogonal to the central axis C of the gas injection port GP. That is, the end face of the cover portion located closest to the negative direction X2 side of the end faces of the cover portions that face the gas injection port GP only need to be located outward of the edge of the gas injection port GP in the direction orthogonal to the central axis C of the gas injection port GP.
In the embodiment described above, the first dimension H1 and the second dimension H2 may be smaller than the maximum dimension of the gas injection port GP. In addition, the first dimension H1 and the second dimension H2 may be the same as or different from each other. In addition, the first dimension H1 and the second dimension H2 may be equal to or larger than the tolerance between the cover member 120 and the nozzle body 110. For example, the first dimension H1 may be 0.07 mm or more. In addition, the same applies to the second dimension H2.

### Addition

The technical concept that can be understood in accordance with the embodiment and the modification described above will be described.

[1] A spray nozzle comprising: a nozzle body connected to a pump; and a cover member, attachable to and detachable from the nozzle body the cover member, that covers an outer surface of the nozzle body, wherein the outer surface of the nozzle body and an inner surface of the cover member define a suction passage through which a liquid circulates, the nozzle body defines a gas injection passage through which a liquid pressurized and fed from the pump circulates and a liquid ejection passage connected to the suction passage on a downstream side, in a cross section including a gas injection port that is an opening of the gas injection passage on a downstream side and a liquid ejection port that is an opening of the liquid ejection passage on a downstream side, when a direction from the gas injection port to the liquid ejection port is a positive direction and a direction opposite to the positive direction is a negative direction, the cover member includes a cover portion, in contact with, on a positive direction side, an upper surface of the nozzle body facing the positive direction side, that is exposed to an outside, and, of end faces of the cover portion that face the gas injection port, an end face located closest to a negative direction side is located outward of an edge of the gas injection port in a direction orthogonal to the positive direction.

[2] The spray nozzle according to [1], further comprising: a liquid pool between the suction passage and the liquid ejection passage, wherein a maximum flow path sectional area of the liquid pool is larger than a maximum flow path sectional area of the suction passage.

[3] The spray nozzle according to [2], wherein the liquid pool is defined by the outer surface of the nozzle body and the inner surface of the cover member.

[4] The spray nozzle according to any one of [1] to [3], wherein the nozzle body has, as the upper surface, a first upper surface, disposed at the same position as an end in the positive direction of the gas injection port in a direction along the positive direction, that is orthogonal to the positive direction, and the cover portion includes a first cover portion in contact with the first upper surface.

[5] The spray nozzle according to any one of [1] to [4], wherein the nozzle body has, as the upper surface, a second upper surface located on the positive direction side of the liquid ejection passage, and the cover portion includes a second cover portion in contact with the second upper surface.

[6] The spray nozzle according to any one of [1] to [5], wherein entire areas of the end faces of the cover portion that face the gas injection port are located outward of the edge of the gas injection port in the direction orthogonal to the positive direction.

[7] The spray nozzle according to any one of [1] to [6], wherein, when a virtual straight line is drawn so as to intersect the positive direction through an end in the negative direction of the gas injection passage and an end of the upper surface closest to the gas injection port, entire areas of the end faces of the cover portion that face the gas injection port are located outward of the virtual straight line in the direction orthogonal to the positive direction.

What is claimed is:

1. A spray nozzle comprising:
a nozzle body connected to a pump; and
a cover member attachable to and detachable from the nozzle body, the cover member covering an outer surface of the nozzle body,
wherein the outer surface of the nozzle body and an inner surface of the cover member define a suction passage through which a liquid circulates,
the nozzle body defines a gas injection passage through which a liquid pressurized and fed from the pump circulates, and a liquid ejection passage connected to the suction passage on a downstream side,
in a cross section including a gas injection port being an opening of the gas injection passage on the downstream side and a liquid ejection port being an opening of the liquid ejection passage on the downstream side, when a direction from the gas injection port to the liquid ejection port is a positive direction and a direction opposite to the positive direction is a negative direction, the cover member includes a cover portion, the cover portion being in contact with, on a positive direction side, an upper surface of the nozzle body facing the positive direction side, and being exposed to an outside, and
of end faces of the cover portion facing the gas injection port, an end face located closest to a negative direction side is located outward of an edge of the gas injection port in a direction orthogonal to the positive direction.

2. The spray nozzle according to claim 1, further comprising:
a liquid pool between the suction passage and the liquid ejection passage,
wherein a maximum flow path sectional area of the liquid pool is larger than a maximum flow path sectional area of the suction passage.

3. The spray nozzle according to claim 2,
wherein the liquid pool is defined by the outer surface of the nozzle body and the inner surface of the cover member.

4. The spray nozzle according to claim 1,
wherein the nozzle body has, as the upper surface, a first upper surface, disposed at a same position as an end in the positive direction of the gas injection port in a direction along the positive direction, and being orthogonal to the positive direction, and
the cover portion includes a first cover portion in contact with the first upper surface.

5. The spray nozzle according to claim 1,
wherein the nozzle body has, as the upper surface, a second upper surface located on the positive direction side of the liquid ejection passage, and
the cover portion includes a second cover portion in contact with the second upper surface.

6. The spray nozzle according to claim 1,
wherein entire areas of the end faces of the cover portion facing the gas injection port are located outward of the edge of the gas injection port in the direction orthogonal to the positive direction.

7. The spray nozzle according to claim 1,
wherein, when a virtual straight line is drawn to intersect the positive direction through an end in the negative direction of the gas injection passage and an end of the upper surface closest to the gas injection port,
entire areas of the end faces of the cover portion facing the gas injection port are located outward of the virtual straight line in the direction orthogonal to the positive direction.
